(19) United States
(12) Patent Application Publication
(10) Pub. No.: US 2015/0140647 A1
(43) Pub. Date: May 21, 2015

(54) SYSTEM FOR THE EXECUTION, TRACEABILITY, MONITORING AND CONTROL OF A METHOD OF REDUCING THE BACTERIAL COUNT IN A CONFINED ENVIRONMENT

(71) Applicant: Matteo LONGO, Bairo (Torino) (IT)

(72) Inventor: Matteo Longo, Bairo (IT)

(21) Appl. No.: 14/411,831

(22) PCT Filed: Jul. 3, 2013
(86) PCT No.: PCT/IB2013/055449

§ 371 Date: Dec. 29, 2014

(30) Foreign Application Priority Data
Jul. 4, 2012 (IT) TO2012A000589

(51) Int. Cl. C12Q 1/22; A61L 9/14

(52) U.S. Cl. CPC C12Q 1/22; A61L 9/14
USPC 435/287.4

(57) ABSTRACT

A system (1) for the execution, traceability, monitoring and control of reducing the bacterial count in a confined environment, includes an environment identification device (2) arranged to contain information relating to the confined environment. A device (3) for micronizing air-dispersed decontaminant substances is arranged to diffuse through a diffuser (9) a decontaminant substance (12) by air as dry fog, based on the information contained in the environment identification device (2). A detection sensor (4) detects the concentration of the decontaminant substance (12). An instantaneous bacterial analysis sensor (5) determines the quantitative and qualitative bacterial concentration. A central server (6) processes data received from the diffuser (9), from the detection sensor (4) and from the instantaneous bacterial analysis sensor (5), to identify anomalies with respect to a desired quantitative and qualitative bacterial objective and certify the bacterial result obtained as a result of the diffusion of the decontaminant substance (12).

The present invention relates to a system for the execution, traceability, monitoring and control of a method of reducing the bacterial count in a confined environment, such as a laboratory or a clean room.

In particular, the present invention relates to a system for monitoring the use of apparatus, devices and decontaminant substances for the execution of operating procedures for the purpose of quantitative and qualitative bacterial maintenance of any confined environment.

At the present time, the quantitative and qualitative bacterial level in a confined environment is maintained by the use of a number of predetermined manual operating procedures, such as cleaning and the removal of organic material present in the confined environment and on the surfaces located therein, the disinfection or sanitization of these surfaces by the application of decontaminant chemical products, and the rinsing and drying of said surfaces.

The monitoring of the operational activities of sanitization and disinfection of the aforesaid confined environments is therefore carried out manually by an operator, and is documented in a record or “self-monitoring plan” which may be accompanied by qualitative bacterial sampling conducted by qualified external operators on the air and surfaces of the confined environment.

It follows from the above that the monitoring activity cannot be validated scientifically and automatically, owing to the large number of manual operations that it requires; instead, the operator employed to perform these operations signs a simple documented self-certification of the activities performed.

This very commonly gives rise to errors of transcription, while also entailing a risk of bacterial exposure for the operator, with repercussions on the operator's health and on the planned activity within the confined environment.

Object of the present invention is therefore to propose a system for the execution, traceability, monitoring and control of a method of reducing the bacterial count in a confined environment which enables the risk of human error in the activities of disinfection and sanitization of the confined environment to be reduced or eliminated, in order to ensure the attainment of an expected quantitative and qualitative bacterial objective, while providing automatic and error-free traceability of each individual operation relating thereto and certifying the bacterial result obtained.

These and other objects are achieved by means of a system for the execution, traceability, monitoring and control of a method of reducing the bacterial count in a confined environment, the principal characteristics of which are defined in claim 1.

Specific embodiments are described in the dependent claims, the content of which is to be considered as an integral and essential part of the present description.

Further characteristics and advantages of the present invention will be made clear by the following detailed description, provided purely by way of non-limiting example, with reference to the attached drawings, in which:

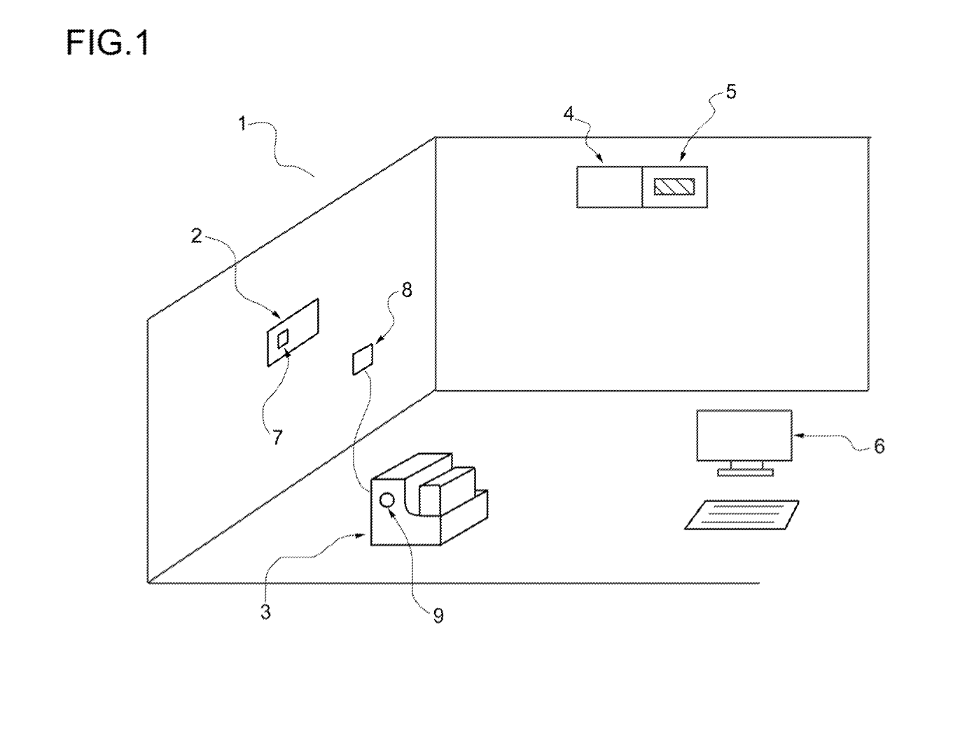

FIG. 1 is a perspective front view of the system according to the present invention, applied in a confined environment;

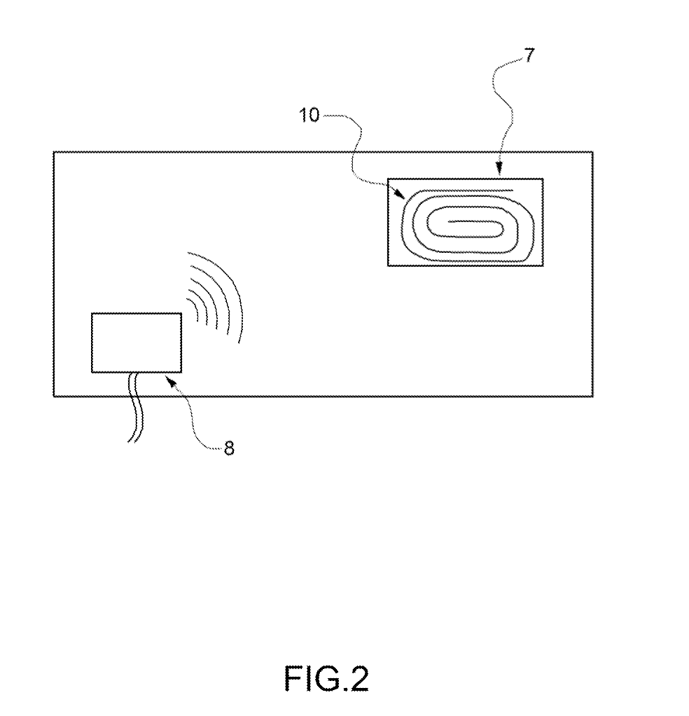

FIG. 2 is a front view of the environment identification sensor of the system according to the invention;

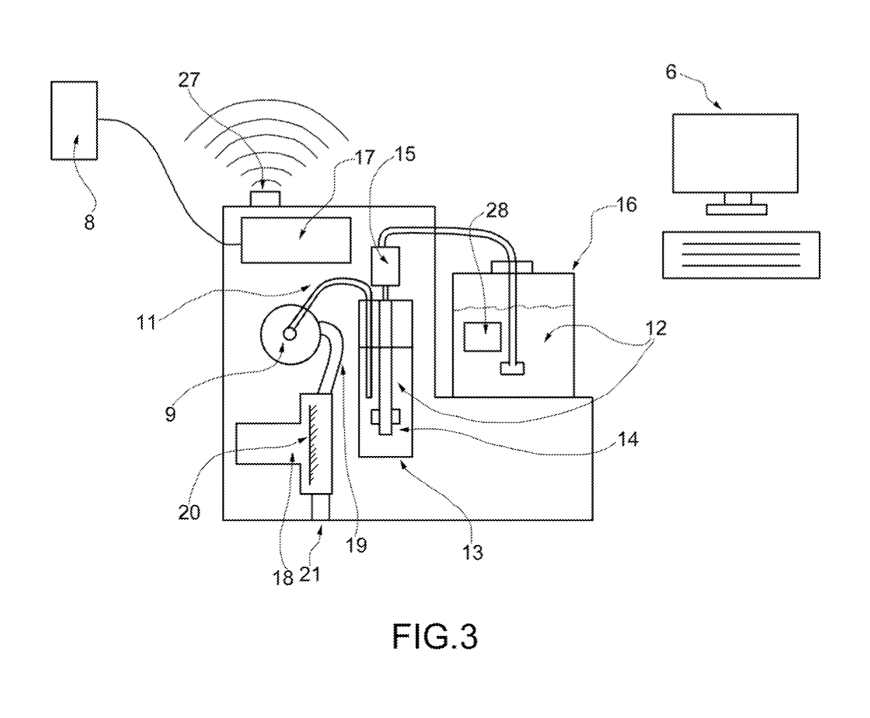

FIG. 3 is a front view of the device for micronizing decontaminant substances;

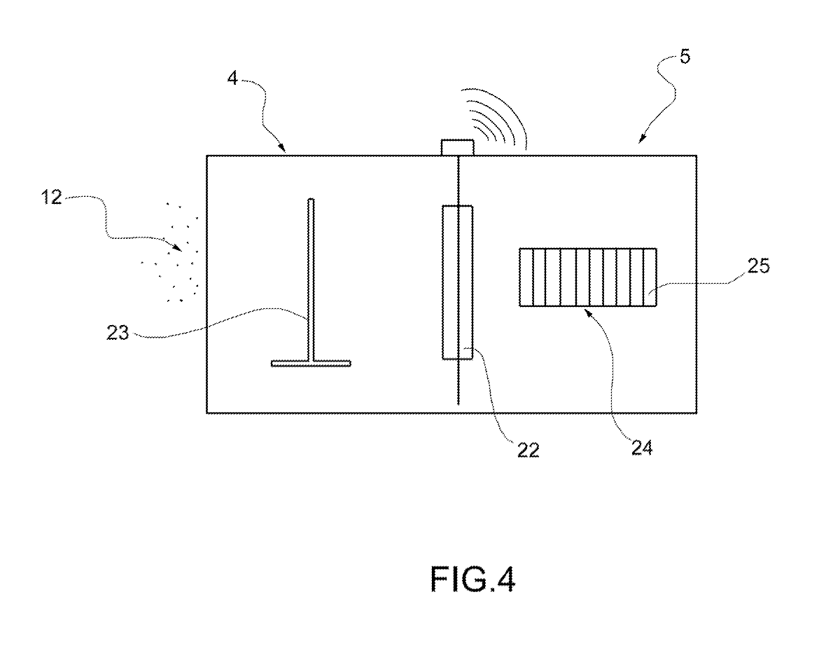

FIG. 4 is a front view of the detection sensor and the instantaneous bacterial analysis sensor.

Briefly, the system according to the invention can be used to enable and control, by acquiring all the technical and architectonic data of each individual confined environment for which bacterial quality maintenance is required, the activity of a device for micronizing decontaminant substances present in the confined environment.

The micronizing device distributes a decontaminant substance uniformly within the confined environment, without leaving residues on the surfaces, and calibrates its activity of bacterial count reduction (decontamination) in order to achieve an expected quantitative and qualitative bacterial objective.

This method of reducing the bacterial count in a confined environment is applied in conformity with the modes of use specified in the technical and safety data sheets for the decontaminant substances used and in the user's manuals of the micronizing device. The bacterial count reduction in a confined environment is carried out with respect to quantitative and qualitative parameters predetermined by the users, to allow the certification of the bacterial result obtained.

The system according to the invention identifies all the disinfection and sanitization operations carried out within the confined environment and the type of decontaminant substances used for the purpose of achieving the expected objective.

By using an instantaneous bacterial analysis sensor, activated before or at the end of the bacterial count reduction method, the system of the present invention is capable of carrying out immediate bacterial quantity and quality sampling and providing official certification of the result obtained.

The acquired information can be used for the creation of a database, the processing of statistical parameters and the analysis of the costs and other information relating to the activity of qualitative environmental maintenance, for each individual confined environment.

In FIG. 1, the number 1 indicates a system for the execution, traceability, monitoring and control of a method of reducing the bacterial count according to the present invention.

This system 1 comprises an environment identification device 2, preferably fixed within the confined environment, and containing known storage means 7, containing the technical information relating to the method of reducing the bacterial count and the characteristics of the confined environment. In particular, the storage means 7 contain data representative of the periodicity of execution of the decontamination treatment.

The system 1 further comprises a micronizing device for micronizing air-dispersed decontaminant substances 3, comprising a transponder reader 8 for acquiring information about the confined environment to be treated (supplied by the environment identification device 2) and a diffuser 9 adapted to produce decontaminant substances in the form of dry fog.

The transponder 8 is also arranged to send to a central server 6 data relating to the method of reducing the bacterial count, such as the quantity of decontaminant substance delivered, the delivery time, and the like, said central server 6 coordinating the activity of the various devices of the system 1.

The data acquired by the central server 6 are processed in a known way by the server 6 itself so as to identify anomalies with respect to the expected objective and certify, by creating a “self-monitoring plan”, the bacterial result obtained.

The processing of the data provides, in a known way, for the creation of graphs, the processing of operating protocols for prevention, the analysis of the costs of the process executed, and any other information useful for the improvement of the bacterial conditions of the confined environment, for the purpose of providing a virtual technical and scientific file on the confined environment.

The micronizing device 3 is capable of self-calibration on the basis of the information received from the environment identification device 2 and the data relating to the decontaminant substance, acquired for example by means of the transponder 8 arranged to read a code present on the packaging of the decontaminant substance.

Finally, the system 1 comprises a detection sensor 4 arranged to detect the concentration of the decontaminant substance delivered into the confined environment by the diffuser 9.

The system 1 further comprises an instantaneous bacterial analysis sensor 5 capable of determining the exact quantitative and qualitative bacterial level before and after the use of the method of reducing the bacterial count.

At the end of each cycle of bacterial count reduction, the micronizing device 3 operates in a known way to transfer the information on the executed activity to the central server 6. The micronizing device 3 also sends to the instantaneous bacterial analysis sensor 5 a signal to start the step of analysis of the qualitative and quantitative bacterial level present in the confined environment.

In turn, the instantaneous bacterial analysis sensor 5 sends the results of the completed analysis to the central server 6.

FIG. 2, in which parts and elements identical or corresponding to those of FIG. 1 have been given the same reference numerals, shows the environment identification device 2, comprising the memory means 7 containing a writable printed circuit 10 into which are input all the architectonic and volumetric information on the confined environment and the technical information relating to the method for reducing the bacterial count, such as the planned periodicity of treatment, thus determining the optimal concentration of product to be delivered.

FIG. 3, in which parts and elements identical or corresponding to those of FIG. 1 have been given the same reference numerals, shows the micronizing device 3 in which the diffuser 9 diffuses decontaminant substances through the air and in the form of dry fog. The micronizing device 3 operates by the Venturi effect: a decontaminant substance 12 is drawn through a delivery tube 11 from a collecting reservoir 13, the level of which is kept constant by means of a float switch 14 connected to a squeeze pump 15 which fills the reservoir 13 by drawing the decontaminant substance 12 from the original retail pack 16. The decontaminant substance 12 is identified in respect of its organoleptic characteristics by means of a printed circuit positioned in a descriptive label 28 present on the pack 16.

The activity of all the components of the system according to the invention is controlled by a control unit 17 which, following the acquisition of the data on the confined environment by the transponder 8, starts the operation of an electric motor 18 connected to a blower 19 capable of proportionally mixing the decontaminant substance 12 with the quantity of air drawn in from the external environment by a fan 20 driven by the motor 18. The diffuser 9, operating by means of a data transmission system (not shown in the drawing), of the Bluetooth, SMS or GPRS type for example, transmits all the information about its own activity to the central server 6 for subsequent processing as described above.

FIG. 4, in which parts and elements identical or corresponding to those of FIG. 1 have been given the same reference numerals, shows the detection sensor 4 for detecting the concentration of the decontaminant substance, comprising a piezoelectric probe 23 which is connected, through a microprocessor 22, to an instantaneous bacterial analysis sensor 5 based on a microfluidic platform of the lab-on-chip type 24 comprising sensitive microstructures 25 in the form of a “cantilever” or “array of cantilevers”, arranged to capture any pathogenic microorganisms and to signal to the microprocessor 22 the quantity of microorganisms detected and their characteristics.

The microprocessor 22 is adapted to transfer the acquired information relating to the activity of the detection sensor 4 and the instantaneous bacterial analysis sensor 5, by means of a data transmission system of the Bluetooth, SMS or GPRS type 26 for example, to the central server 6 for subsequent processing as described above.

Advantageously, the system according to the present invention is capable, by means of the input into the central server 6 of qualitative and frequency parameters relating to activities included in the operational “self-monitoring plan” for a confined environment, of generating, on a preventive basis or following the discovery of contamination, reports of non-conformity of data and alarm signals which are transmitted in real time to the persons in charge of the operations and activities concerned.

Advantageously, the system according to the invention is capable of producing, by means of the systematic processing of the data obtained from the activity of the devices, statistical graphs and evaluation parameters for the costs compared with the direct and indirect benefits obtained in relation to the quality maintenance activity, while certifying the self-monitoring process specified in advance.

The method applied by the system according to the present invention makes it possible to achieve an optimal quality standard for a given confined environment, while avoiding procedures of in loco bacterial sampling by external operators and the consequent deterioration of bacterial quality found as a result of the previous decontamination process. For this purpose, it is no longer necessary to carry out operations of collecting and transporting sampled material in order to determine the bacterial level of the confined environment.

Clearly, provided that the principle of the invention is retained, the forms of application and the details of embodiment can be varied widely from what has been described and illustrated purely by way of non-limiting example, without thereby departing from the scope of the invention as defined in the attached claims.

1. System for the execution, traceability, monitoring and control of a method of reducing the bacterial count in a confined environment, comprising:
an environment identification device arranged to contain information relating to the confined environment;
a device for micronizing air-dispersed decontaminant substances arranged to diffuse through a diffuser a decontaminant substance by air as dry fog, based on the information contained in the environment identification device;
a detection sensor adapted to detect concentration of the decontaminant substance;
an instantaneous bacterial analysis sensor arranged to determine quantitative and qualitative bacterial concentration;
a central server arranged to process data received from the diffuser, from the detection sensor and from the instantaneous bacterial analysis sensor, to identify anomalies with respect to a desired quantitative and qualitative bacterial objective and certify a bacterial result obtained as a result of the diffusion of the decontaminant substance.

2. System according to claim 1, wherein the environment identification sensor comprises memory containing technical information relating to the method for reducing the bacterial count in a confined environment.

3. System according to claim 1, wherein the micronizing device comprises:
a collecting tank adapted to contain the decontaminant substance;
a tube arranged to draw the decontaminant substance from the collecting tank;
a fan arranged to draw air from the environment to send said air to a blower arranged to mix said air with the decontaminant substance received from the collecting tank;
a motor adapted to drive the fan.

4. System according to claim 3, wherein the collecting tank contains a float switch connected to a squeeze pump arranged to fill the collecting tank with the decontaminant substance drawn from a retail pack so as to maintain a predetermined level of the decontaminant substance in the collecting tank.

5. System according to claim 1, wherein the detection sensor comprises a piezoelectric probe connected, though a microprocessor, to the instantaneous bacterial analysis sensor.

6. System according to claim 1, wherein the instantaneous bacterial analysis sensor is based on a lab-on-chip microfluidic platform comprising sensitive microstructures in the form of a “cantilever” or “array of cantilevers”, arranged to capture pathogenic microorganisms and to signal to the microprocessor a quantity of microorganisms detected and their characteristics.
